(19) United States
(12) Patent Application Publication
(10) Pub. No.: US 2010/0010127 A1
(43) Pub. Date: Jan. 14, 2010

(54) Oleochemical Plasticizers with Thermal and Ultraviolet Radiation Stabilizing Activity for PVC Molding Resins and Process for Obtaining Thereof

(75) Inventors: Andres Cohen Barki, Estado de Mexico (MX); Nicolas Ramirez-de-Arellano-Aburto, Atizapan de Zaragoza (MX); Aldimir Torres-Arenas, Cuautitlan Izcalli (MX); M. Javier Cruz-Gomez, Colonia Roma Sur. (MX)

Correspondence Address: STERNE, KESSLER, GOLDSTEIN & FOX P.L.L.C., 1100 NEW YORK AVENUE, N.W., WASHINGTON, DC, 20005, US

(73) Assignee: Resinas y Materiales, S.A. de C.V.

(21) Appl. No.: 12/485,781

(22) Filed: Jun. 16, 2009

(30) Foreign Application Priority Data
Jun. 17, 2008 (MX) MX/A/2008/007817

(51) Int. Cl. C08K 5/1515; C07D 301/02

(52) U.S. Cl. 524/114; 549/518

(57) ABSTRACT

The present invention is related with bioplasticizers or primary oleochemical plasticizers and the improved process for obtaining thereof. It refers primarily to epoxydized oleochemical plasticizers produced from vegetable oils, as substitute of traditional petrochemical plasticizers. The process starts with the epoxydized product of natural oils, such as sunflower, linseed, Jatropha curcas, soybean, etc., which are transesterified with an alcohol such as ethylic or methylic, in the presence of a catalyst such as sodium methoxide or sodium hydroxide in order to produce an alkylic esters mixture of the fatty acids that were present in the oil or oil mixture used as raw material in the epoxydized oil production. When the plasticizer obtained by the process already mentioned is used for the formulation of moldable poly(vinyl chloride), PVC, resins; the resulting plastic films get adequate hardness, static and dynamic thermal stability, and plasticizer extractability by solvents, such as n-hexane, gasoline and oil. Besides, when the PVC resin is formulated with a phthalic or terephthalic plasticizers mixture and the bioplasticizer, the bioplasticizer presents a full range solubility and or compatibility with the remainder of the resin compounds. The oxyrane chemical ring of the bioplasticizer is an excellent chemical neutralizer of the HCL that might be formed from the PVC, due to the action or interference of thermal or UV radiation.

### BACKGROUND OF THE INVENTION

1. Field of the Invention

The present invention is located on the chemistry field, particularly it is related to an improved process for the plasticizers production of the type known as oleochemical or bioplasticizer obtained from vegetal oils, such as sunflower, linseed, palm, Jatropha curcas, rape seed, soybean, among others. All of these oils have the chemical characteristic of being triesters of unsaturated fatty acids and glycerin.

For the present invention, the oil or the chosen oil mixture are epoxydized using any already known industrial processes, characterized by the use of an organic acid, such as formic or acetic acid, with hydrogen peroxide, or a peracid such as the peracetic or similar. Depending on the selected oil, there is obtained an epoxydized product with a characteristic oxyrane number range. Thus when sunflower oil or soya natural oil are selected, there is obtained a product with an oxyrane number between 6.0 and 7.2; with linseed oil, the epoxydized product has an oxyrane number between 8.0 and 9.5 and with winterized soya oil, there is obtained a product with an oxyrane number between 6.5 and 7.5. When an oil mixture is selected, there is obtained an oxyrane number proportional to the oil mixture used. A transesterification reaction is carried out between the epoxydized oil, or oil mixture and a light alcohol such as ethanol or methanol, using catalytic amounts of sodium methoxide or sodium hydroxide. One of the reaction products are glycerin, which is separated as the bottom phase by decantation, and a methylic or ethylic esters mixture of the fatty acids contained in the oil or oil mixture used as starting raw material.

When methanol is used, as light alcohol, in the manufacture of the bioplasticizer, or final product, there have been identified by applying IR spectroscopy, gases-masses chromatography and proton and C-13 nuclear magnetic resonance, the following chemical substances: methyl stearate, methyl 9-10-epoxyoctadecanoate, methyl 9-10,12-13-diepoxyoctadecanoate, methyl 9-hydroxi, 12-13-epoxyoctadecanoate, methyl 9-10,12-13,15-16-triepoxyoctadecanoate. All of these chemicals came from the chemical transformation process, which took place during the bioplasticizers production, from the glyceric esters of the stearic, oleic, linoleic, linolenic and other similar acids. These esters are the vegetal oil components from the starting raw materials. By the same techniques and for the same reason, methyl palmitate and myristate are as well identified, but in a lower proportion.

It was found that the epoxydized fatty-acids alkylic-esters bioplasticizer has excellent primary plasticizer properties and behaves as thermal and UV radiation stabilizer as well. When the oleochemical plasticizer, object of this invention, is used for the poly(vinyl chloride) mold-resin formulations; the plastic films, produced from this resins, give satisfactory results with respect to the hardness, dynamic and static thermal stability, and plasticizer extractability to solvents such as n-hexane, gasoline and oil. Besides, when the resin formulation is prepared with a different phthalic or terephthalic plasticizer mixture, or with special plasticizers that give stain resistance; the oleochemical plasticizers present a full range solubility and/or compatibility with the other poly(vinyl chloride) resin components.

2. Background Art

The plasticizer is the main additive mixed with the poly(vinyl chloride), PVC; to transform it into a processable resin. Alkyl phthalates and terephthalates are commonly used as primary PVC plasticizers. PVC resins usually contain variable amounts of secondary plasticizers, also called thermal or UV light stabilizers, such as a mixture of barium, cadmium and zinc soaps, organic phosphates and/or soybean or linseed epoxy oils; besides other different additives such as, pigments, reinforcements, fillers, etc. All of this is described by: Wilson, J. E. Stabilizers for vinyl resins, U.S. Pat. No. 2,707,178, (1955); DiBella, Eugene P. Stain-resistant plasticizer composition and method of making same, U.S. Pat. No. 5,153,342, (1992); and Nass, I. Leonard, Encyclopedia of PVC, Marcel Dekker, Inc. New York (1977), p. 651 and 852.

PVC is produced with different molecular weights related with the resin final use. A low molecular weight PVC is easy to process, but has the lower values for the resin qualification parameters; in the other hand, a high molecular weight has outstanding properties, but it is difficult to process. The plasticizer mixed with a relatively high molecular weight PVC has as a function the improving of the PVC resin processability. In industrial practice, (as it is referred by Coelho, Jorge F. J., Gonçalves, Pedro M. F. O., Miranda, D. and Gil, M. H. Characterization of suspension poly(vinyl chloride) resins and narrow polystyrene standards by size exclusion chromatography with multiple detectors: Online right angle laser-light scattering and differential viscometric detectors. European Polymer Journal 42 (2006) 751-763), the K-value is used to give an idea of PVC molecular weight. The PVC K-values between 50 and 85 correspond to PVC number average molecular weights between 25 and 100 Kilodaltons. By the processing of the PVC resin, there are manufactured a variety of human commodities, such as films for packaging materials, industrial and domestic piping, shoe sole, bottles, toys, isolated wire, etc.

Dioctyl phthalate (DOP) and dioctyl terephthalate (DOTP) are the most commonly used PVC plasticizers, although sometimes, other phthalic or terephthalic alkyl esters are also used as PVC plasticizers. All these plasticizers are manufactured with raw materials derived from the crude oil. But nowadays, with the prices of crude oil continuously rising, due to the gradual exhausting of this non-renewable resource; and the expectation that the crude oil prices behavior will be in the next decades similar to what has been in the last two decades; it is visualized as a reasonable alternative the production, from natural oils, a renewable resource, of the present epoxydized oleochemical plasticizer.

From all the above mentioned, it is concluded that there is the necessity of an alternative plasticizer manufactured from renewable raw materials, and presenting as well a high plasticizing efficiency over high molecular weight PVC.

So it is the objective of the present invention to provide an improved process, for the production of a plasticizer from vegetable oils, or an epoxydized oleochemical plasticizer, characterized by including the epoxidation, light alcohol transesterification, glycerin by-product separation and final product neutralization steps.

Other objective of the present invention is to provide another option for plasticizers raw materials which come from a different source than crude oil, because crude oil is a non-renewable raw material.

Other objective of the present invention is to provide an improved chemical process for the production of an epoxydized oleochemical plasticizer, or bioplasticizer, made from renewable raw materials, such as natural oils.

Other objective of the present invention is the epoxydized oleochemical plasticizer, or bioplasticizer, that can be used in the PVC resin formulation, and when the polymer films made from this resin are tested, they give satisfactory results with respect to hardness, static and dynamic thermal stability, and plasticizer extractability to solvents such as n-hexane, gasoline and oil.

Another objective of the present invention is the epoxydized oleochemical plasticizer, or bioplasticizer, that when the resin formulation is prepared with a different phthalic or terephthalic plasticizers mixture, or with special plasticizers such as the ones that give stain resistance; the oleochemical plasticizers present a full range solubility and/or compatibility with the other PVC resin components.

### DESCRIPTION OF THE INVENTION

The present invention refers to the process for obtaining epoxydized oleochemical plasticizers from natural oils, mainly oils of the safflower, sunflower, linseed, palm, Jatropha curcas and soybean type, among others; either alone or in a mixture. In accordance with the present invention, the oils in question are epoxydized by means of any of the already known industrial processes. They use an organic acid with hydrogen peroxide or a per-acid such as peracetic acid or other similar ones. According to the present invention, the reactive and/or catalysts excess are removed at the reaction end, through washing followed by separation of the aqueous bottoms. This action assures the plasticizers purity in order to avoid any interference between impurities and the PVC resin.

The present process, object of this invention begins with oil that has already been epoxydized or with an epoxydized mixture of unsaturated organic acids esterified with glycerin. The primary raw material is epoxydized oil, with a preferred oxyrane number between 6.0 and 7.2 when soya or sunflower oil is used and a preferred oxyrane number between 8.0 and 9.5 in the case of linseed oil.

According to the present invention, methyl or ethyl alcohol is added over the epoxydized oil contained in the transesterification reactor, the catalyst should be dissolved in the alcohol. One mole of sodium methoxide or sodium hydroxide is dissolved per each 20 to 100 moles of ethanol or methanol.

In table 1 are shown the different process parameters that have been used in the development of the present invention. The bench scale batches were carried out with 1 kg of epoxydized oil as starting raw material. For the pilot plant batches it was used 50 kg, and for the industrial batches it was used 20 to 24 metric tons of epoxydized oil as starting raw material.

TABLE 1
Parameters used in plasticizer manufacture, from epoxydized oil

| | Lab-01 | Pil-02 | Ind-03 | Ind-04 |
|---|---|---|---|---|
| Epoxydized oil, kg | 1.030 | 50.0 | 23,000 | 21,000 |
| Methanol, kg | 0.180 | 8.74 | 4,050 | 3690 |
| Sodium hydroxide, kg | 0.0052 | 2.48 | 115 | 105 |
| Oxalic acid, kg | 0.002 | — | — | 45 |
| Formic acid, Kg | — | 0.67 | 31 | — |
| Plasticizer produced, kg | 0.980 | 48.5 | 22,480 | 20,520 |
| Glycerin obtained, kg | 0.112 | 6.070 | 2,792 | 2,150 |
| Recovered Methanol, kg | 0.010 | 1.5 | 720 | 665 |
| Reaction time, min | 90 | 50 | 100 | 80 |
| Reaction temperature, ° C. | 58 | 60 | 59 | 60 |

The next procedure is followed in order to obtain the plasticizers of the present invention. In a batch reactor, provided with systems of agitation, heating, cooling, vacuum, and attached to a reflux column, it is prepared a mixture of epoxydized oil and catalyzed alcohol. It was used 0.5 to 0.7 alcohol moles per each 100 grams of epoxydized oil. The mixture is continuously stirred. The reaction temperature is in the range of 50 and 80 centigrade degrees. The reaction times were in the range of 20 to 80 minutes. When reaction time is completed, the stirrer is stopped and the system is leaved in repose at a temperature of 40° C. to 60° C., in order to let settle down the glycerin dense phase. As soon as the glycerin phase is separated, the basicity of the product inside the reactor is neutralized by adding formic or oxalic acid dissolved in water. The product hydrogen potential should reach a value between 6 and 8. Once again, the system is leaved in repose, but this time to settle down and remove an aqueous phase. A neutral product is left in the reactor. Vacuum is applied to the system, which is heated up to a temperature between 80 and 110 centigrade degrees for a period of time of half an hour. During this step it is recovered ethylic or methylic alcohol and water, which may still be dissolved and/or suspended in the oily system. The dried product is cool down to room temperature in order to get the final epoxydized oleochemical plasticizer.

The oleochemical plasticizer was characterized in the laboratory. Table 2 shows the results of this characterization applied to the products obtained with the four batches described in Table 1.

TABLE 2
Parameters obtained when the bioplasticizer is obtained from
epoxydized oil

| | Lab-01 | Pil-02 | Ind-03 | Ind-04 |
|---|---|---|---|---|
| Acid Index, mg KOH/g | 0.85 | 0.76 | 1.26 | 1.14 |
| Density, g/cc | 0.955 | 0.948 | 0.953 | 0.954 |
| Viscosity, cp. | 23 | 24.6 | 24.3 | 22.5 |
| Color, Apha | 130 | 120 | 140 | 120 |
| Iodine Index, I₂/100 g | 1.33 | 1.02 | 0.66 | 0.8 |
| Oxyrane Index, wt % | 6.83 | 6.73 | 6.66 | 6.56 |
| Humidity, wt % | 0.083 | 0.094 | 0.064 | 0.043 |

With the epoxydized oleochemical plasticizers prepared according to batches 1 to 4, of the Tables 1 and 2, three PVC resin samples were prepared. Their formulation is shown in columns 4 to 6 of Table 3. In order to have reference standards, there were prepared PVC resin samples, but now using dioctyl phthalate (DOP), as plasticizer instead of epoxydized oleochemical plasticizer. The formulation for these resins is shown in columns 1 to 3 of Table 3. The PVC used for all six resin samples is a standard product. It is described as the homopolymer: poly(vinyl chloride), produced in suspension, with a K-value of 65, which corresponds to an average number molecular weight of 48,000 gram/mol and polydispersity of 2. (Coelho, Jorge F. J., Gonçalves, Pedro M. F. O., Miranda, D. and Gil, M. H. Characterization of suspension poly(vinyl chloride) resins and narrow polystyrene standards by size exclusion chromatography with multiple detectors: Online right angle laser-light scattering and differential viscometric detectors. European Polymer Journal 42 (2006) 751-763).

TABLE 3
Parameters used for the formulation of K65 value PVC resins

| | Formulation No. | | | | | |
|---|---|---|---|---|---|---|
| Material | 1 | 2 | 3 | 4 | 5 | 6 |
| PVC resin, | 100.0 | 100.0 | 100.0 | 100.0 | 100.0 | 100.0 |
| K65 | | | | | | |
| Dioctyl | 30.0 | 50.0 | 70.0 | — | — | — |
| phthalate | | | | | | |
| Bioplasticizer | — | — | — | 30.0 | 50.0 | 70.0 |
| Thermal | 0.5 | 0.5 | 0.5 | 0.5 | 0.5 | 0.5 |
| stabilizer, | | | | | | |
| Ba/Zn | | | | | | |
| Stearic acid | 0.35 | 0.35 | 0.35 | 0.35 | 0.35 | 0.35 |

With the 6 PVC resins, prepared according to Table 3 formulations, there were molded different films to analyze thermal and mechanical properties, extractability and plasticity. The obtained results are shown in Table 4.

TABLE 4
Comparative tests of PVC resins plasticized with DOP and
bioplasticizer

| | Formulation No. | | | | | |
|---|---|---|---|---|---|---|
| Material | 1 | 2 | 3 | 4 | 5 | 6 |
| Hardness, Shore A | 95 | 82 | 73 | 95 | 78 | 70 |
| Fusion time in ETD, min:seg | 23:56 | 59:40 | 59:22 | 3:40 | 33:38 | 3:32 |
| Decomposition time in ETD, | 59:40 | 59:44 | 59:32 | 59:38 | 59:40 | 59:32 |
| min:seg | | | | | | |
| Film resistance to solvent | 15.96 | 27.30 | 35.42 | 15.36 | 26.06 | 34.24 |
| extraction, weight %. | | | | | | |
| Film resistance to oil | 1.09 | 4.00 | 12.54 | 2.12 | 9.75 | 19.86 |
| extraction, weight %. | | | | | | |
| Film resistance to a gasoline | 10.68 | 21.70 | 31.40 | 8.38 | 22.47 | 30.97 |
| extraction (Naphtha), | | | | | | |
| weight %. | | | | | | |
| Plasticizer lost at 80° C., | 1.30 | 0.34 | 1.09 | 1.11 | 1.94 | 1.57 |
| during 24 hours, weight %. | | | | | | |

The hardness of PVC resins prepared with epoxydized oleochemical plasticizer is similar to the resins prepared with DOP. The dynamic thermal stability (DTS) analysis, show that the fusion time is less when the resin is prepared with bioplasticizer instead of DOP; they also show that the decomposition time is similar for the resins prepared with both kinds of plasticizers. Extraction tests show that PVC resins prepared with epoxydized oleochemical plasticizer present a lower extractability with solvent and gasoline, and a high extractability with oil, as compared to resins prepared with DOP as plasticizer.

About the oil extractability, it is important to mention that the oils used for the four batches of Table 1 are natural oils, which contain saturated oils, besides the unsaturated oils susceptible to be epoxydized. But, if for some application it is required an oil extractability of the PVC resin films similar to the one obtained with DOP, this can be achieved by preparing the bioplasticizer with winterized oils instead of natural oils. The so called winterized oils are characterized by the lack of saturated oils, due to the separation of them, after they were subjected to cold temperatures. The epoxydized oleochemical plasticizer prepared with winterized oils is free of the methyl esters of saturated acids. The resins prepared with winterized oil bioplasticizer have oil extractability similar to the resins prepared with DOP. It was also found that the PVC resins, prepared with winterized oil bioplasticizer, have a good anchoring for impression ink and a good adherence to the different industrial adhesives used with PVC films.

The six PVC resins, prepared according to Table 6, were also used to carry out the static thermal stability analysis. This is a visual test that allows the comparison of the yellowness level that PVC films achieve, when these films are placed in an oven at 180 centigrade degrees. The exposure time is from 10 to 60 minutes. The results of the visual tests show that the resins prepared with epoxydized oleochemical plasticizer have a better behavior than the ones plasticized with DOP.

Resins 4 to 6 from tables 3 and 4, by having as plasticizer the bioplasticizer object of the present invention, which contains several oxyrane rings in its molecules, have the chemical property of been able to act as HCl neutralizing agent. Hydrogen chloride is formed inside the resin by UV and/or thermal degradation of poly(vinyl chloride), PVC. Weather inclemency's, to which the PVC films are subjected during its life time, are the direct cause of the chemical degradation experienced by the different commodities or final products manufactured with PVC.

1. A process for the production of epoxydized oleochemical plasticizers, comprising the following steps:
performing vegetal oil epoxidation using an organic acid with oxygenated water, or, a peracid, in order to obtain the respective epoxydized oils;
performing a transesterification reaction between the epoxydized oil or oil mixture, and a light alcohol, in the presence of catalytic amounts of sodium methoxide or sodium hydroxide;
performing glycerin separation, as inferior phase by decantation;
performing neutralization of a methyl or ethyl ester mixture of fatty acids from starting raw materials; and
performing final product drying, in order to obtain the epoxydized oleochemical plasticizer.

2. A process for the production of epoxydized oleochemical plasticizers according to claim 1, wherein the epoxydized oil or oil mixture has an oxyrane number between 6.0 and 7.2 for pure epoxydized soy bean oil and between 8.0 and 9.5 for pure epoxydized linseed oil.

3. A process for the production of epoxydized oleochemical plasticizers according to claim 1, wherein the transesterification reaction between the light alcohol and the epoxydized oil or oils mixture is carried out by the dissolution of one mole of sodium methoxide or sodium hydroxide per 20 to 100 moles of ethanol or methanol.

4. A process for the production of epoxydized oleochemical plasticizers according to claim 3, wherein the transesterification reaction is carried out with 0.5 to 0.7 moles of light alcohol per each 100 grams of epoxydized oil, at a temperature between 50 and 80 Celsius degrees, during a reaction time of 20 to 100 minutes.

5. A process for the production of epoxydized oleochemical plasticizers according to claim 1, wherein after the by-product glycerin is separated, a reaction product is neutralized with formic acid, oxalic acid, or a combination of formic acid and oxalic acid, until a preferred hydrogen potential between 6 and 8 is obtained.

6. A process for the production of epoxydized oleochemical plasticizers according to claim 5, wherein the reaction product is vacuum dried in order to recover the methanol or ethanol and moisture present in the system.

7. A process for the production of epoxydized oleochemical plasticizers according to claim 1, wherein the epoxydized oil is a natural oil or a mixture of selected oils.

8. A film comprising:
PVC molding resins manufactured using epoxydized oleochemical plasticizers obtained by the process of the claim 1, wherein the film has improved properties of hardness, static and dynamic thermal stability, ultraviolet radiation resistance and solvent extractability, when n-hexane, gasoline and cooking oil are used as solvents for extractions.

9. Epoxydized oleochemical plasticizers obtained by the process of the claim 1, wherein the epoxydized oleochemical plasticizers have bioplasticizers oxyrane chemical rings that are an effective neutralizing agent, against HCl produced by thermal and or ultraviolet degradation of PVC products when the epoxydized oleochemical plasticizers are used in PVC resin formulations.

10. Epoxydized oleochemical plasticizers obtained by the process of the claim 1, wherein the epoxydized oleochemical plasticizers have a full range of compatibility and solubility with formulation components of PVC resins.

11. A process for the production of epoxydized oleochemical plasticizers according to claim 2, wherein after the by-product glycerin is separated, a reaction product is neutralized with formic acid, oxalic acid, or a combination of formic acid and oxalic acid, until a preferred hydrogen potential between 6 and 8 is obtained.

12. A process for the production of epoxydized oleochemical plasticizers according to claim 3, wherein after the by-product glycerin is separated, a reaction product is neutralized with formic acid, oxalic acid, or a combination of formic acid and oxalic acid, until a preferred hydrogen potential between 6 and 8 is obtained.

13. A process for the production of epoxydized oleochemical plasticizers according to claim 4, wherein after the by-product glycerin is separated, a reaction product is neutralized with formic acid, oxalic acid, or a combination of formic acid and oxalic acid, until a preferred hydrogen potential between 6 and 8 is obtained.

14. A process for the production of epoxydized oleochemical plasticizers according to claim 11, wherein the reaction product is vacuum dried in order to recover the methanol or ethanol and moisture present in the system.

15. A process for the production of epoxydized oleochemical plasticizers according to claim 12, wherein the reaction product is vacuum dried in order to recover the methanol or ethanol and moisture present in the system.

16. A process for the production of epoxydized oleochemical plasticizers according to claim 13, wherein the reaction product is vacuum dried in order to recover the methanol or ethanol and moisture present in the system.
